(19) United States
(12) Patent Application Publication
(10) Pub. No.: US 2003/0175365 A1
(43) Pub. Date: Sep. 18, 2003

(54) Natural drug induced differentiation cancer cells to resemble normal cells

(76) Inventor: Yaguang Liu, Queens, NY (US)

Correspondence Address: Yaguang Liu, 67-08 168th Street, Flushing, NY, 11365, US

(21) Appl. No.: 10/097,753

(22) Filed: Mar. 15, 2002

(51) Int. Cl. A61K 35/78

(52) U.S. Cl. 424/725

(57) ABSTRACT

The new pharmaceutics preparation of HHT derivates and HHT-derivates containing sterically stabilized liposomes (HHT-SSL) in accordance with the present invention are for inducing differentiation cancer cells to resemble normal cells and apoptosis of cancer cells. Anticancer molecular mechanism of HHT-SSL and HHT includes inhibiting oncogenes, increasing activity of tumor suppressor, inhibiting cancer cells proliferation, inhibiting DNA methylation, and increasing activity of APO-2L.

### BACKGROUND OF THE INVENTION

The present invention related to natural drug for treating and preventing cancer by induced differentiation of cancer cells to resemble normal cells and inhibited proliferation and apoptosis of cancer cells.

Specifically, this invention provides a safe drug homoharringtonine derivates and its new preparation.

### DESCRIPTION OF THE PRIOR ART

A great development in the oncology, enabled by progression of molecular and cellular technologies throughout the culminating in 1990s was transformed from killing both cancer and normal cells to induce differentiation cancer cells to resemble normal cells and do not injure the normal cells.

Cancer is the second leading cause of death in the United States, and the incidence of cancer continues to climb annually. In recent years, about 1 million new cases of cancer are diagnosed yearly in the U.S. About half million people and 7 million people of annual deaths are in the US and in the world, respectively. A lot of anticancer drugs including chemical and antibiotics have effects to kill cancer cells. But it also kills off some normal human cells, appears many kinds of side effects, among them the inhibition of bone marrow and tract are the most common.

Cyclopho-sphamide, for example, is a chemotherapeutic drug, which is highly effective against a wide range of human cancer. Cyclophosphamide established a role in the treatment of some major cancer types including Lymphomas, Acute Lymphatic leukemia, Chronic Lymphatic Leukemia, Breast, Pulmonal, Ovarial cancer and Tumor or marrow mulciple, Osseous, Sarcoma etc. Unfortunately, Cyclophospharmide has high toxicity, for example, it does damage to hemotopoietic organs, alimentary tract and decrease immune function. The toxicity of other anti-cancer medicines, for example, Fluorouracil, Mustine and 6-Mercaptopurine, etc. is higher than Cyclophosphamide.

Some antibiotics are effective anticancer drugs, for example, Adriamycin is used for treatment of some cancers including leukemia, gastric pancreatic, breast cancer, etc. A prime limit factor to the administration of Adriamycin is cardiotoxicity. The most serious side effect of Adriamycin administration is myocardial degeneration causing congestive heart failure. This acute cardiomyopathy may cause acute left ventricular dysfunction, arrhythmia and myocardial infarction. Adriamycin induced cardiomyopathy is thought to be permanent and rapidly progressive. Late cardiomyopathy develops in weeks, months or even years. Some patients were reported to have developed progressive cardiomyopathy two and two-half years after receiving this drug.

Obviously, to overcome toxicity of anti-cancer is very important. Many agents have been suggested to reduce or prevent toxicity of anticancer medicines. For example, Vitamin E and N-acetyl-L-Cysteine have also been reported to be effective in preventing Adriamycin cardiotoxicity. Although more recent research showed evidence to contradict these findings, in that neither of vitamin E and N-acetyl-L-Cysteine prevents adriamycin-induced cardiotoxicity.

The major antiviral drugs can inhibit viral replication but also inhibit some host cell function and possess serious toxicity. For example, Amantadine, Idoxuridine, Cytarabine, and Vidarabine are major antiviral drugs using in clinic now. Amantadine can inhibit influenza A.

The most marked toxic effects of Amantadine are insomnia, slurred speech, dizziness, ataxia and other central nervous system sign. Idoxuridine can inhibit the replication of herpes simplex virus in the cornea. However, DNA synthesis of host cells is also inhibited. Cytarabine can inhibit DNA viruses. But it also inhibits immune function in human. By weight it is about 10 times more effective than Idoxuridine, and it is also 10 times more toxic for host cell. Vidarabine can inhibit herpes-virus, but it is also produces more marked adverse gastrointestinal or neurologic side effects.

Many reports indicated that the side effects of plant's anticancer drugs are lower than chemical and antibiotic's anticancer drugs. Therefore, the development of plant drug has progressed very fast now. Taxol, for example, is a novel anticancer plant drug isolated from the needles and bark of the western yew, Taxus brevifolia. It is the prototype for a new class of antitumor drugs, which are characterized by their capacity to promote the assembly of microtubules. Clinical trials conducted in the late 1980s and early 1990s demonstrated impressive clinical activities against advanced ovarian and breast cancer. Taxol, however, still has some side effects, for example, myelosuppression, diarrhea, emesis, oligospermia, cellular depletion in lymphoid tissues, changes in serum hepatic enzymes, and elevations in cholesterol and triglycerides were observed. More important, taxol has two big problems. The first problem is that natural source of taxol is very limited. And the second problem is that taxol is a poor-water soluble. Vehicles for parental administration on taxol cause serious side effects. Natural drug, which used for treating and preventing cancer by induced differentiation of cancer cells to resemble normal cells and inhibited proliferation and apoptosis of cancer cells, is very important.

### DETAILED DESCRIPTION OF THE INVENTION

In the present invention experiments show that HHT induced differentiation of cancer cells, including leukemia cells and gastric cancer cells to resemble normal cells. HHT also inhibits growth and induced apoptosis of cancer cells. Induced differentiation and apoptosis is important goal of cancer therapy.

The following specific examples will provide detailed illustrations of methods of producing relative drugs, according to the present invention and pharmaceutical dosage units containing demonstrates its effectiveness in treatment of cancer cells. These examples are not intended, however, to limit or restrict the scope of the invention in any way, and should not be construed as providing conditions, parameters, reagents, or starting materials which must be utilized exclusively in order to practice the present invention.

### EXAMPLE 1

Effect of HHT on Differentiation of Human Leukemic Cells

Methods

Cell Lines. HL-60 cells were established from a patient with acute myeloid leukemia. The cells were cultured in culture flasks with RPMI plus 10% FCS.

Studies of Induction of Differentiation. Differentiation of HL-60 cells was assessed by their abilities to produced superoxide as measured by reduction of NBT, by NSE staining and by morphology as detected on cytospin preparations stained with Diff-Quick stain Set, and by analysis of membrane-bound differentiation markers with two-color immunofluorescence. Briefly, cells were preincubated at 4° C. for 60 min in 10% human AB serum and then with FITC-conjugated mouse IgGI isotype control. Analysis of fluorescence was performed on a flow cytometer.

Cell Cycle Analysis. The cell cycle was analyzed by flow cytometry after 60 h of incubation of HL-60 cells either with or without HHT (10⁻⁸ M) as described. Briefly, the cells were fixec in cold methanol and incubated for 30 min at 4° C. in the dark with a solution of 50 μg/ml propidium iodide, 1 mg/ml Rnase, and 0.1% NP40. Analysis was performed immediately after staining using the CELLFIT program whereby the S-phase was calculated with Rfit model.

Clonogenic Assay in soft Agar. HL-60 cells were culture in a two-layer soft agar system for 10 days without adding any growth factors as described previously, and colonies were counted using an inverted microscope. The analogues were added to the agar upper layer on day 0. For analysis of the reversibility of inhibition of proliferation, the cells were cultured in suspension culture with and without HHT. After 60 h, the culture flasks were gently jarred to loosen adherent cells, the cells were washed twice in cultured medium containing 10% FCS to remove the test drugs, and then the clonogenic assay was performed.

These results were periodically confirmed by fluorescence microscopy and by DNA fragmentation.

TABLE 1
Effect o HHT on cellular differentiation of leukemic cells

| Group | NBT (%) | NSE (%) |
|---|---|---|
| Normal cells | 98 | 97 |
| Leukemic cells | 5 | 6 |
| Leukemic cells + HHT | 65* | 62* |

*P < 0.001 compared with group of leukemic cells.

Data of Table 1 showed that HHT could significantly induce differentiation of leukemic cells.

### Example 2

Effect of HHT on Differentiation of Gastric Cancer Cells

The gastric cancer cells and normal cells were cultured in PRMI 1640 medium supplement with 10% FCS serum. Other method is similar to example 1.

TABLE 2
Effect of HHT on differentiation of gastric cancer cells

| Group | NBT (%) | NSE (%) |
|---|---|---|
| Normal gastric cells | 95 | 92 |
| Gastric cancer cells | 8 | 5 |
| Gastric cancer cells + HHT | 58* | 60* |

*P < 0.001 compared with group of leukemic cells.

Data of Table 2 showed that HHT could significantly induce gastric cancer cells.

### Example 3

Effect of HHT on Differentiation of Epidermis Cancer

It is known that tetradecanoyphorbol-13-acetate (TPA) is strong tumor promoter and TPA can remarkable increase [³H] thymidine incorporation in mouse epidermis and then to induce Epidermis cancer.

Methods M

ice were treated with TPA and the rate of [³H] thymidine incorporation was determined 20 hours later. Male mice (7-9 weeks old) used for experiments. Only mice showing no hair regrowth following shaving were used. Animals were injected intraperitoneally (i.p.) with TPA or 95% saline. After 20 hours, mice were injected i.p. with 60 μCi of [³H] thymidine (2 Ci/mmol) 20 minutes before sacrifice. Epidermal scrapings were prepared. Epidermis cancer cells were cultured in condition of example 1.

TABLE 3
Effect of HHT on differentiation of epidermis cancer

| Group | NBT (%) | NSE (%) |
|---|---|---|
| Normal epidermis cells | 97 | 96 |
| Epidermis cancer cells | 3 | 2 |
| Epidermis cancer cells + HHT | 80* | 78* |

*P < 0.001 compared with group of leukemic cells.

Data of Table 3 indicated that HHT can remarkable inhibit DNA synthesis of TPA-stimulated mouse epidermis. Therefore, the experiments indicated that HHT could induce differentiation of epidermis cancer cells.

### EXAMPLE 4

Effect of HHT and HHT-SSL on Apoptosis of Cancer Cells

Methods

Human leukemia cells (HL-60) were grown in RPMI Medium 1640 supplemented with 10% (v/v) heat-inactivated FBS (56° C. for 30 min) at 37° C. in a humidified 95% air/5% CO₂ atmosphere. Cells were seeded at a level of 2×10⁵ cells/ml. Cells were allowed to attain a maximum density of 1.2×10⁶ cells/ml before being passed by dilution into fresh medium to a concentration of 2×10⁵ cells/ml.

Apoptosis Determined by two Methods:

Method (1): Cell pellets containing 5×10⁶ cells were fixed with 2.5% glutaraldehyde in cacodylate buffer (pH 7.4), dehydrated through graded alcohol, and infiltrated with LX-112 epoxy resin. After overnight polymerization at 60°C. 1-μm sections were cut with glass knives using a LKB Nova microtome. The sections were stained with 1% toluidine blue and coverslipped. In addition, experimental examples were stained with May-Grunwald-giemsa stain for the demonstration of apoptosis.

DNA electrophoresis: Untreated and treated HL-60 cells collected by centrifugation, washed in phosphate buffered saline and re-suspended at a concentration of 5×10^{6 cells and} 0.1% RNase A. The mixture was incubated at 37° C. for 30 min and then incubated for an additional 30 min at 37° C. with 1 ml protease K. Buffer was added and 25 μl of the tube content transferred to the Horizontal 1.5% agarose gel electrophoresis was performed at 2 V/cm. DNA in gels visualized under UV light after staining with ethidium Bromide (5 μg/ml). DNA fragmentation assays: DNA cleavage was performed, quantitation of fractional solubilized DNA by diphenylamine assay and the percentage of cells harboring fragmented DNA determined by in labeling techniques. For the diphenylamine assay, 5×10⁶ cells were lysed in 0.5 mL lysis buffer (5 mmol/L Tris-HCl, 20 mmol/L DTA, and 0.5% Triton X-100, pH 8.0) at 4° C. Lysates were centrifuged (15,000 g) for separation of high molecular weight DNA (pellet) and DNA cleavage products (supernatant). DNA was precipitated with 0.5 N perchloric acid and quantitated using diphenylamine reagent. The cell cycle distribution was determined 4 hours after addition of drug and represents mean ±SD of 5 independent experiments.

Method (2): Apoptosis of HL-60 cells was assessed by changes in cell morphology and by measurement of DNA nicks using the Apop Tag Kkt (Oncor, Gaithersburg, Md). Morphologically, HL-60 cells undergoing apoptosis possess many prominent features, such as intensely staining, highly condensed, and/or fragmented nuclear chromatin, a general decrease in overall cell size, and cellular fragmentation into apoptotic bodies. These features make apoptotic cells relatively easy to distinguish from necrotic cells. These changes are detected on cytospin preparations stained with Diff-Quick Stain Set. Apoptotic cells were enumerated in a total of about 300 cells by light microscopy.

For evaluation of apoptosis by flow cytometry, cells were fixed and permeabilized in 1% paraformaldehyde and ice-cold 70% ethanol. Digoxigenin-dUTP was incorporated at the 3′OH ends of the fragmented DNA in the presence of terminal deoxynucleotidyltranserase, and the cells were incubated with FITC-labeled anti-digoxigenin-dUTP and with propidium iodide. Green (apoptotic cells) and orange (total DNA) fluorescence were measured with a FACScan flow cytometer and analyzed with LYSIS II and CELLFIT programs.

TABLE 4
Effect of HHT-SSL and HHT on apoptosis of cancer cells

| Drug | |
|---|---|
| Concentration (μm) | Apoptosis (%) |
| Control | 0 |
| HHT (20) | 68.3 ± 7.6 |
| HHT (20)-SSL | 85 ± 9.5* |

*P < 0.001 compared with group of leukemic cells.

Data of Table 4 indicated that HHT-SSL and HHT could significantly induce apoptosis and HHT-SSL is better than HHT.

### EXAMPLE 5

HHT Extraction

HHT was extracted from the skins, stems, leaves and seeds of Cephalotaxus fortunei Hook and other related species, such as Cephalotaxus sinensis Li, C. hainanensis, and C. wilsoniana. 1 kg of ground Cephalotaxus fortunei Hook was extracted with 8 liters of 90% ethanol at room temperature for 24 hrs. Filtered the solution to yield a filtrate A and filtercake. Percolated the filtercake with ethanol and filter again to yield filtrate B. Combined A and B, and distilled under reduced pressure to recover ethanol and an aqueous residue. To this residue, added 2% HCl to adjust the pH to 2.5. Separated the solids from the solution by filtration to yield a filtrate C. Washed the solids once with 2% HCl and filtered to yield a filtrate D. Combined C and D and adjusted the pH to 9.5 by adding saturated sodium carbonate solution. Extracted the alkaline filtrate with chloroform and separated the chloroform layer from the aqueous layer. Repeated this extraction process five times. Combined all the chloroform extracts and distilled at reduced pressure to recover chloroform and alkaloid as a solid residue respectively. The solid alkaloid was then dissolved in 6% citric acid in water. The solution was divided into three equal portions. These were adjusted to pH 7, 8 and 9 by adding saturated sodium carbonate solution. The portions having pH 8 and 9 were combined and extracted with chloroform. The chloroform extracts were distilled under reduced pressure, whereby chloroform was removed and recovered and crude HHT was obtained. The crude HHT was dissolved in pure ethanol and crystallized. The crystals were refined by recrystallization in diethyl ether.

The portion having a pH of 7 passed through a liquid chromatographic column packed with alumina of diameter to height 1:50. The column was finally flushed with chloroform and followed by chloroform-methanol of 9:1 mixture. The resulting alkaloids were mixture of HHT. The mixed HHT was then separated from each other by countercurrent distribution employing chloroform and pH 5 buffers. The first fraction of the countercurrent distribution was HHT. HHT was purified by crystallization in methyl alcohol.

HHT has the following chemical structure:

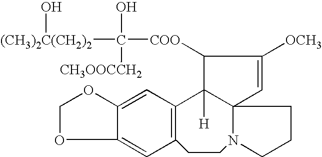

Yield: 0.02%.

Melting point: 144°-146° C.

Infrared spectrum: 3500, 3400, 1665, 1030 and 940 cm-.

Ultraviolet spectrum: λpeak alcohol mμ (logμ): 240 (3.55), 290 (3.61).

### EXAMPLE 6

Preparation of HHT-Containing Sterically Stabilized Liposomes (HHT-SSL)

Hydrogenated phosphatidylcholine (PC), phosphatidylglycerol (PGL), and phosphatidylserine (PS) were extracted from soybean. All above lipids were finally purified on silicic acid columns, shown to be pure by thin-layer chromatography and stored in chloroform in sealed ampules under nitrogen until use. Phospholipids mixed with cholesterol (CHOL) and long-chain alcohol. The solvent was removed under reduced pressure by a rotory evaporator. The lipids were then purged with nitrogen. Lipids were redissolved in the organic phase and reversed phase will be formed. HHT-containing phosphate-buffered saline (HHT was 3 mM in 0.1 M phosphate-buffered saline) was added at these lipid systems, and resulting two-phase system was sonicated 3 minutes until the mixture homogeneous that did not separate for at least two hours after sonicated. A typical preparation contained 3.3×10⁻³ M of phospholipid and 3.3×10⁻³ M of cholesterol in 1 litre of phosphate-buffered saline and 3 litre of solvent. HHT-SSL were sealed and sterilized. [³H]-HHT and dialyzed method was used to determine the amount of encapsulated HHT. The size of the vesicles was determined by a dynamic light cattering technique. When PG/PC/CHOL were 1:4:5, diameter of liposomes was 20-50 nM (range). HHT-SSL was very stabilized in at least six months.

So far, many articles reported drug-containing liposomes. However, liposomes are not stabilized. In general methods, liposomes are stabilized in one month or less. Therefore it is difficult to be used for pharmaceutical industry. In accordance with this invention, HHT-SSL is very stabilized in at least six months. Therefore HHT-SSL can be used in industry. HHT-SSL can enhance cancer targeting and improve anticancer activity of HHT. It is very important that all lipids are extracted from soybean. Therefore it is very safe for human being. Many articles reported lipids, which used for drug-containing liposomes, are syntheses by organic chemistry. However synthetic lipids have some side effects, therefore, methods of preparing of HHT-SSL and phospholipids, which extracted from soybean, are very safe.

### EXAMPLE 7.

Effect of HHT and HHT-SSL on Regulation of Oncogenes

Human myeloblastic leukemic cells (ML-1) were maintained in suspension culture in RPMI 1640 medium supplemented with 7.5% heat-inactivated FBS. Cells growth and viability were assayed by hemocytometer using trypan-blue dye exclusion.

RNA was isolated by the CsCl gradient modification. RNA pellets were washed twice by reprecipitation in ethanol and quantitated by absorbency at 260 nM. RNA analyzed by electrophoresis of 15 μg of RNA through 1.2% agarose formaldehyde gels followed by northern blot transfer to nitrocellulose.

Single-standard uniformly labeled DNA probes were prepared. Probe of c-myc was a 1.7 Kb cla-Eco RI restriction fragment containing the 3′exon region of human c-myc and probe of c-myb was 1.0 Kb myb-specific Bam HI fragment. Probes for n-ras contained DNA fragments using a modification of the PCR technique. Probes for myb, myc and n-ras were isolated. The isolated fragments were labeled to high specific activity with [β³²]-dCTP (3000 ci/mmol). Prehybridization of the filter was performed. The hybridization mixer contained 50,000 cpm of probe. The probes were hybridized at 58° C. in 15 mM NaCl, 1.5 nM sodium citrate for 3 hours. After hybridization, they were exposed to XAR-5 film. Oncogene expression was quantitated by densitometer scanning of the autoradiography. The results are summarized in the tables as below

TABLE 5
The effect of HHT and HHT-SSL concentration on inhibition of
oncogenes

| HHT concentration | Inhibition (%) | | |
|---|---|---|---|
| (ng/ml) | c-myb RNA | c-myc RNA | P |
| 0 | 0 | 0 | — |
| 10 | 65.0 ± 5.7 | 70.5 ± 8.5 | <0.01 |
| 50 | 67.7 ± 6.8 | 70.8 ± 8.9 | <0.01 |
| HHT-SSL | 66 ± 7.0 | 72.8 ± 7.5 | <0.01 |
| HHT-SSL | 72 ± 7.5 | 75 ± 8.4 | <0.001 |

nM = 10⁻⁹ M

This study clearly indicated that HHT-SSL and HHT could significantly inhibit oncogenes of cancer cells and HHT-SSL is better than HHT. Cellular oncogenes encode proteins have important function in differentiation of cancer cells. The principal functions of c-myc are the induction of proliferation and the inhibition of terminal differentiation in many cells. Over-expression of myc commonly occurs in a wide range of tumors. EXAMPLE 8

Effect of HHT on Tumor Suppressor p⁵³

The recent progress made in molecular biology has revealed that tumor suppressor p⁵³ is a tumor suppressor. Disorder of mutations of p⁵³ plays a very important role in the development of many cancers. ¹⁷p allelic and p⁵³ mutations appears to inhibit cancer growth. However, determinate tumor suppressor of cancer cells is very difficult and experimental errors are lager. The present invention proved a new and easy method for determinate tumor suppressor of cancer cells.

Methods

The leukemic cancer cells and normal cells were cultured in RPMI 1640 medium supplement with 10% fetal bovine serum. All the exons of the p⁵³ were amplified by the polymease chain reaction (PCR) using specific oligonucleotide primers. The PCR products were subjected to single-strand conformation polymorphism (SSCP) analysis. A second PCR-SSCP analysis was performed to ensure that the results were reproducible in each experiment, which showed mobility. Levels of DNA methylation were determined.

Results

TABLE 6
Effect of HHT on p⁵³ mutations

| | Frequency of p⁵³ | Inhibition T/C | |
|---|---|---|---|
| Group | mutations (%) | (%) | P |
| Normal gastric cells | 0 | 0 | — |
| Gastric cancer cells (no drug) | 35 | — | — |
| Gastric cancer cells treated | 5 | 14.3 ± 1.5 | <0.01 |
| by HHT | | | |

Data of Table 6 showed that HHT could obviously inhibit levels of p⁵³ mutations of cancer cells. It means that HHT could increase function of tumor suppressor. Increased tumor suppressor could treat and prevent cancer. EXAMPLE 9

Effect of HHT on Tumor Suppressor p¹⁷

The p¹⁷ is a tumor suppressor. The gastric cancer cells were used in this experiment. Gastric cancer cells and normal cells were cultured in RPMI 1640 medium supplement with 10% fetal bovine serum. All the exons of tumor suppressor p¹⁷ were amplified by the polymease chain reaction (PCR) using specific oligonucleotide primers. The PCR products were subjected to single-strand conformation polymorphism (SSCP) analysis. A second PCR-SSCP analysis was performed. Levels of DNA methylation were determined.

TABLE 7
The effect of Drug on p allelic loss

| | Frequency of p¹⁷ | Inhibition | |
|---|---|---|---|
| Group | allelic loss (%) | T/C (%) | P |
| Normal gastric cells (N) | 0 | 0 | — |
| Leukemic cancer cells (C) | 40 | — | — |
| Leukemic cancer cells treated | 5 | 12.5 ± 1.2% | <0.01 |
| by HHT | | | |

Data of Table 7 showed that HHT obviously inhibits p¹⁷ allelic loss of cancer cells. Therefore, data of Table 6 and 7 showed that HHT could obviously increase activity of tumor suppressor. EXAMPLE 10

Effect of HHT on Tumor Suppressor p¹⁶

The p¹⁶ was the key regulators of the progression of eukaryotic cells through the G1 phase of the cell cycle. The high frequency of p¹⁶ alteration showed in tumor cells.

Methods

Bladder cell lines were used. Methods of RT-PCR were regular. PCR-based methylation determined by inability of some restriction enzymes to cut methylated sequences methods of determination was used to analyze the methylaton status of the p¹⁶ and p¹⁵ . PCR products were resolved on 2% agarose gels transferred to a nylon membrane and hydridized for the RT-PCR products. An undigested DNA control and MSPI-digested DNA control were examined. The results are listed as the following table.

TABLE 8
Methylation of tumor suppressor p¹⁶

| Group | Methylation (%) |
|---|---|
| Normal cells (N) | 15.8 |
| Cancer cells (no treatment) (C) | 81.5^{a} |
| Cancer cells treated by HHT (T) | 23.8^{b} |

^{a}P < 0.001, different with normal group
^{b}P < 0.01, different with no treatment group

The p¹⁶ tumor suppressor might be inactivated by methylation. Methylation of p¹⁶ might be a mechanism to regulation of expression of this tumor suppressor. Data of Table 8 indicated that HHT inhibited methylation of p¹⁶. HHT could increase active of tumor suppressor. HHT, therefore, could prevent or treat cancer.

### EXAMPLE 11

Effects of HHT and HHT-SSL on Tumor Cells Proliferation

Materials and Methods

Human tumor cell lines: Hela leukemia HL-60, malignant melanocarcinoma B16, oral epidermoid carcinoma (KB), lung carcinoma (A549), breast carcinoma MCF-7, adenocarcinoma of stomach.

Animal tumor cell lines: Walker carcinoma, LLC-WRC-256, malignant melanoma (RMMI 1846), 3T3,and S-180 sarcoma (CCRF-180). All lines were routinely cultured in the RPMI1640 medium supplemented 20% fetal calf serum. The experiment was carried out in 96 microplate, each well had 5×10⁵ cells and given desired concentration of 1 μg/ml (1 x 10⁻⁶ g/ml) drug. Then the plate was incubated at 37° C. in an atmosphere of humidified air enriched with 5 percent carbon dioxide for 72 hours.

Inhibition percent rate of tumor cell proliferation was obtained according to the bellow formula.

$$Inhibition percent rate = \frac{Control - Test}{Control} \times 100 \%$$

Results

HHT-SSL and HHT inhibited tumor cells growth significantly. Percent rates of inhibition were all more than 70% in all cancer cells by HHT-SSL and HHT. Also HHT-SSL is better than HHT.

TABLE 9
Effect of HHT-SSL and HHT on inhibiting growth cancer cells

Inhibition (%)

| Cell line | HHT | HHT-SSL |
|---|---|---|
| Control | — | — |
| Human cells | | |
| HL-60 | 80.8 ± 9.0 | 85 ± 9.0 |
| Hela | 75.6 ± 8.0 | 82 ± 8.5 |
| B16 | 73.8 ± 8.1 | 79 ± 8.7 |
| KB | 80.0 ± 8.5 | 91 ± 9.8 |
| MCF-7 | 78.5 ± 7.9 | 90 ± 9.9 |

### EXAMPLE 12

Effect of HHT on APO-2L

APO-2L has been shown to induce apoptosis of cancer cells. Mechanism of APO-2L is very complex. In present study, HHT was investigated for relationship among APO-2L, apoptosis and HHT.

Methods

Neuroblastoma tumor cells, melanocytic and epithelial cells used in this experiment. Cells were grown routinely as monolayers in a 1:1 mixture of Eagle's minimum essential medium and Ham's nutrient mixture F-12 supplemented with 15% heat-inactivated fetal calf serum. Cells were used over a maximum of 15 passages to minimize intersubline differentiation.

Cytotoxicity Assays. Etoposide sensitivity was measured by clonogenic assay. Five hundred cells were plated per 2-cm² dish. Cells were treated for 1 hour with HHT or medium. Results are the mean ±S.E. of three independent experiments and duplicate plates were used in each individual experiment. Apoptosis was quantified by the filter binding assay.

Immunostaining. Cells were seeded onto eight-chamber slides at approximately 20% confluency and incubated at 37° C. overnight to adhere. They were treated the next day with either HHT or oligonucleotides. After treatment, they were rinsed once with PBS and fixed in methanol/acetone solution (1:1) for 10 min at room temperature. Fixed cells were rinsed twice with PBS then incubated with antibodies diluted in PBS (1:250) with 0.1% fetal calf serum for 1 hour at room temperature. Cells were rinsed with PBS and incubated with Cy-3 conjugated secondary antibody (1:200) for 1 hour at room temperature. Cells were then washed with PBS and DNA stained with Hoescht 33258 (1 μg/ml in PBS) for 5 min. p₅₃ antibodies.

Northern Blotting. Total RNA was isolated using RNAzol B. Total RNA (20 μg) was loaded per lane at formaldehyde gels and separated overnight. Samples were prepared, electrophoresed, and blotted using standard procedures. Transcript levels were determined by phosphor-image analysis. Probes used were MDM2,HindIII fragment of the human cDNA clone FL4; p21^{waf-i/cip-1} insert from pBABE.

Determination of Caspase Activation. The substrate used was Ac-DEVD-AMC, which was made to 5 mM stock in dimethylformamide. On the day of the assay, substrate stock was diluted in water to 500 μM and kept on ice. Cell lysate (20 μg) was diluted to equal volumes (20 μl) in Nonidet-P40 lysis buffer and 1/10th of the volume of substrate was added. Exactly 10 min later, the reaction was stopped by adding the mix into 2 ml of water. Fluorescence was read on a fluorometer at an excitation wavelength of 380 nm and emission wavelength of 460 nm.

TABLE 10
Effect of HHT on apoptosis and APO-2L

| Group | Apoptosis (%) |
|---|---|
| Control | 5.0 ± 0.49 |
| HHT (500 nM) | 39.5 ± 4.1 |
| APO-2L (100 mg/ml) | 20.8 ± 2.0 |
| HHT (500 nM) + APO-2L (100 mg/ml) | 72.8 ± 6.9 |

TABLE 11
Effect of HHT on caspase-3 activity

| Group | Caspase-3 activity |
|---|---|
| Control | 25.4 ± 3.0 |
| HHT | 150.5 ± 16 |

APO-2L, also called TRAIL, has been shown to induce apoptosis of variety of cancer cell types. APO-2L can bind to several members of the tumor necrosis factor receptor family including DR4 and DR5. It is important that DR4 and DR5 as the death domain responsible for transducing the death signal. Binding of APO-2L/TRAIL to DR4 and DR5 leads to induce activity of APO-2L. Processed and activated caspase can also activate the BH3 domain containing the proapoptotic Bid protein, which the translocate to mitochondria, triggering the cytosolic release of cyt c. In the cytosal, cyt c causes its oligonerization, then activates the executioner caspase cleave a number of cellular proteins, eg., polymerase, DNA frag mentation factor and resulting in the morphological features and DNA fragmentation of apoptosis.

The data of Table 10 and 11 mean that APO-2L/TRAIL-induced apoptotic signaling; HHT can obviously increase APO-2L/TRAIL-induced apoptotic signaling.

### EXAMPLE 13

Safety of HHT

LD₅₀: The LD₅₀ of HHT in mice (I.P.) was found to be 3.17±0.19 mg/kg. Toxic doses for dogs: 0.16 mg/kg/day×7 of HHT was established as the toxic doses. Toxic doses for mice: In 38 normal mice after injection of HHT of 2 mg/kg/day×5 with the observation period of 5 days about 50% of the mice died.

LD50 of HHT is much higher than majority anticancer drugs. The toxicology data of HHT mean that HHT is safe drug for treatment of cancer cells.

The preparation of drugs, which can be accomplished by the extraction methods set forth above or any conventional methods for extracting the active principles from the plants. The novelty of the present invention resides in the mixture of the active principles in the specified proportions to produce drugs, and in the preparation of dosage units in pharmaceutically acceptable dosage form. The term “pharmaceutically acceptable dosage form” as used hereinabove includes any suitable vehicle for the administration of medications known in the pharmaceutical art, including, by way of example, capsules, tablets, syrups, elixirs, and solutions for parenteral injection with specified ranges of drugs concentration.

In addition, the present invention provides novel methods for treatment of cancer cells with produced safe pharmaceutical agent.

It will thus be shown that there are provided compositions and methods which achieve the various objects of the invention and which are well adapted to meet the conditions of practical use.

As various possible embodiments might be made of the above invention, and as various changes might be made in the embodiments set forth above, it is to be understood that all matters herein described are to be interpreted as illustrative and not in a limiting sense.

What is claimed as new and desired to be protected by Letter Patent is set forth in the appended claim:

1. A safe natural drug for inducing differentiation cancer cells to resemble normal cells and apoptosis of cancer cells comprises Homoharringtonine (HHT) derivates or HHT-derivates containing sterically stabilized liposomes (HHT-SSL).

2. A safe natural drug for inhibiting oncogenes, increasing activity of tumor suppressor, inhibiting cancer cells proliferation, inhibiting DNA methylation, and increasing activity of APO-2L comprises Homoharringtonine (HHT) derivate or HHT-derivates containing sterically stabilized liposomes (HHT-SSL).

3. A safe natural drug, according to claim 1, wherein said Homoharringtonine derivate is extracted from Cephalotaxus sinensis Li or Cephalotaxus hainanensis Li.

4. A safe natural drug of claim 1 wherein the HHT derivate is Homoharringtonine.

5. A safe natural drug of claim 1 wherein the HHT derivate is Harringtonine.

6. A safe natural drug of claim 1, wherein the amount sufficient to induce differentiation of cancer cells to resemble normal cells, is about 25-200 mg of HHT and HHT-SSL. The diameter of liposomes preferred about 20-50 nM.

7. A safe natural drug of claim 1, wherein the amount sufficient to induce apoptosis of cancer cells, is about 25-200 mg of HHT and HHT-SSL. The diameter of liposomes preferred about 20-50 nM.

8. A safe natural drug of claim 1 wherein the amount sufficient to inhibit oncogenes, is about 25-200mg of HHT and HHT-SSL. The diameter of liposomes preferred about 20-50 nM.

9. A safe natural drug of claim 1, wherein the amount sufficient to increase tumor suppressor activity, is about 25-200 mg of HHT and HHT-SSL. The diameter of liposomes preferred about 20-50 nM.

10. A safe natural drug of claim 1, wherein the amount sufficient, to increase activity of APO-2L is about 25-200 mg of HHT and HHT-SSL. The diameter of liposomes preferred about 20-50 nM.

11. A safe natural drug of claim 1, wherein the amount sufficient, to inhibit DNA methylation, is about 25-200 mg of HHT and HHT-SSL. The diameter of liposomes preferred about 20-50 nm.

12. A safe natural drug of claim 1, wherein the amount sufficient to inhibit cancer cells proliferation, is about 25-200 mg of HHT and HHT-SSL. The diameter of liposomes preferred about 20-50 nM.

13. A safe natural drug, uses for induced differentiation cancer cells to resemble normal cells and apoptosis of cancer cells, comprising HHT-SSL, which has more strong anticancer activity and is more safe than HHT.

14. A safe natural drug in accordance with claim 13 wherein said liposomes contained Hydrogenated phosphatidylcholine (PC), phosphatidylglycerol (PGL), and phosphatidylserine (PS).

15. A safe natural drug in accordance with claim 13 wherein said Hydrogenated phosphatidylcholine (PC), phosphatidylglycerol (PGL), and phosphatidylserine (PS) extracted from soybean.

16. A safe natural drug in accordance with claim 15 wherein said Hydrogenated phosphatidylcholine (PC), phosphatidylglycerol (PGL), and phosphatidylserine (PS) purified on silicic acid columns, shown to be pure by thin-layer chromatography.

17. A safe natural drug in accordance with claim 13 wherein said the amount of encapsulated HHT and HHT-SSL were determined by [³H]-HHT and dialyzed.

18. A safe natural drug in accordance with claim 13 wherein said when PG/PC/CHOL were 1:4:5, diameter of liposomes was about 20-50 nM.

19. A safe natural drug of claim 13, which is tablet or capsule form.

20. A dosage unit according to claim 13 wherein said dosage form is tablet, including in addition pharmaceutical acceptable binder and excipients.

21. A dosage unit according to claim 13 wherein said dosage from is a solution for parenteral injection which includes in addition a liquid vehicle suitable for parenteral administration.

22. A process of a safe natural drug in accordance with claim 13 wherein said producing HHT-containing sterically stabilized liposomes (HHT-SSL) is comprising:
(a) Phosphatidylcholine (PC), phosphatidylglycerol (PGL), and phosphatidylserine (PS) were purified from soybean;
(b) PC, PGL, and PS were purified on silicic acid columns;
(c) PC, PGL, and PS mixed with cholesterol (CHOL) and long-chain alcohol;
(d) Lipids were dissolved in the organic phase and reversed phase would be formed;
(e) HHT solution (HHT 3 mM in 0.1 m phosphate-buffered saline) was added at lipid systems and resulting two-phase system was sonicated 3 minutes; and
(f) HHT-SSL was sealed and sterilized.

23. A process for a safe natural drug in accordance with claim 1 wherein said producing homoharringtonine comprising:
(a) extracting a ground plant selected from the group consisting of Cephalotaxus fortunei Hook, C. sinensis Li, C. hainanensis and C. wilsoniana with 90% ethanol at room temperature for 24 hours;
(b) filtering the above mixture and separating a filtrate A from a filtercake;
(c) percolating the filtercake with ethanol and collecting a filtrate B;
(d) combining filtrates A and B and distilling them under reduced pressure to recover ethanol and an aqueous residue;
(e) adjusting the pH of the residue to 2.5 ;
(f) separating solids from the resulting mixture by filtration to yield a filtrate;
(g) adjusting the pH of the filtrate of step (f) to 9.5 ;
(h) extracting the alkaline solution of step (g) five times with chloroform, combining all the chloroform extracts and distilling them to recover alkaloids;
(i) dissolving the alkaloids in citric acid, dividing the solution into three portions, and adjusting the pH of the three portions to 7, 8, and 9 ;
(j) extracting the portions of pH 8 and 9 with chloroform;
(k) distilling the chloroform extract to yield raw harringtonine;
(l) purifying said harringtonine by crystallizing the same in pure ethanol and recrystallizing the same in diethyl ether;
(m) combining the portion of pH 7 of step (i) and the mother liquors resulting from step (l);
(n) passing the solution of step (m) through a chromatographic column packed with alumina, flushing said column with chloroform and subsequently with a chloroform-methanol mixture to yield a mixture of harringtonine and homoharringtonine; and
(o) separating the homoharringtonine from harringtonine by countercurrent distribution with chloroform and pH 5 buffer.
